(19) United States
(12) Patent Application Publication
(10) Pub. No.: US 2013/0109664 A1
(43) Pub. Date: May 2, 2013

(54) Personal Care Compositions Comprising a Pyrithione and an Iron Chelator

(71) Applicants: James Robert SCHWARTZ, West Chester, OH (US); Charles Winston Saunders, Fairfield, OH (US); Robert Scott Youngquist, Mason, OH (US); Casey Patrick Kelly, Wyoming, OH (US); Jeffrey Kenneth Domsic, Liberty Township, OH (US); Robie Lynn Lucas, Liberty Township, OH (US); Jun Xu, Mason, OH (US)

(72) Inventors: James Robert SCHWARTZ, West Chester, OH (US); Charles Winston Saunders, Fairfield, OH (US); Robert Scott Youngquist, Mason, OH (US); Casey Patrick Kelly, Wyoming, OH (US); Jeffrey Kenneth Domsic, Liberty Township, OH (US); Robie Lynn Lucas, Liberty Township, OH (US); Jun Xu, Mason, OH (US)

(21) Appl. No.: 13/283,890

(22) Filed: Oct. 28, 2011

(51) Int. Cl. A61K 31/555; A61P 17/00

(52) U.S. Cl. 514/188

(57) ABSTRACT

The present invention is directed to a personal care composition comprising an effective amount of a pyrithione or a polyvalent metal salt of a pyrithione; an effective amount of an iron chelator or a material which chelates iron; wherein the combination of the iron chelator and the pyrithione or a polyvalent metal salt of a pyrithione has a fractional inhibitor concentration of less than or equal to 1.

### FIELD OF THE INVENTION

The present invention is directed to personal care compositions comprising a pyrithione or a polyvalent metal salt of a pyrithione and an effective amount of an iron chelator.

### BACKGROUND OF THE INVENTION

Mechanism of action (MOA) of the anti-fungal function of Zinc Pyrithione (ZPT) as an anti-dandruff active agent has demonstrated that iron chelation is a major element of the biological activity. Based on these and similar observations, the use of appropriate iron chelators in combination with ZPT is a key insight to developing significantly more effective anti-dandruff formulas.

Zinc Pyrithione (ZPT) is a common anti-dandruff active used in shampoos and other treatments. Mechanistic work has provided elements of its action such as the importance of chelating iron as an anti-fungal strategy. Other parameters identified that may affect the activity of an iron chelator include hydrophobicity which may be an element for compatibility with ZPT such that relatively low Zn affinity is needed.

By utilizing the new MOA understanding of ZPT to achieve increased anti-fungal activity, the present invention will deliver compositions and products with superior anti-dandruff performance.

### SUMMARY OF THE INVENTION

The present invention is directed to a personal care composition comprising an effective amount of a pyrithione or a polyvalent metal salt of a pyrithione; an effective amount of an iron chelator or a material which chelates iron; wherein the combination of the iron chelator and the pyrithione or a polyvalent metal salt of a pyrithione has a fractional inhibitor concentration of less than or equal to 1.

### BRIEF DESCRIPTION OF THE FIGURES

FIG. 1 is an example process of how to interpret a hypothetical FIC, where all numerical values represent final concentration of the tested chemicals in parts per million.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

The components and/or steps, including those which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

Herein, “personal care compositions” means products for and/or methods relating to treating hair (human, dog, and/or cat), including, bleaching, coloring, dyeing, conditioning, growing, removing, retarding growth, shampooing, styling; deodorants and antiperspirants; personal cleansing; color cosmetics; products, and/or methods relating to treating skin (human, dog, and/or cat), including application of creams, lotions, and other topically applied products for consumer use; and products and/or methods relating to orally administered materials for enhancing the appearance of hair, skin, and/or nails (human, dog, and/or cat); and shaving.

### A. Iron Chelators

In the present invention, iron chelators may have, but not be limited to, the following characteristics:

1. An affinity for iron ions in either the ferrous (iron II) or ferric (III) forms;
2. Materials of Description 1 (above) that have a denticity of two or higher (denticity is the number of groups of a molecule that bind to the iron ion);
3. Chemical descriptions that are a subset of Description 2:

a. Either natural or synthetic (e.g., DFO, DFT) materials;

b. Materials of the following chemical classes:
i. Catechols and phenols
ii. Hydroxamates (desferrioxamine (DFO))
iii. Thiohydroxamates
iv. Hydroxypyridones (CP20, piroctone, ciclopirox, HP-101)
v. Hydroxythiopyridones
vi. Hydroxypyridinethiones
vii. Aminocarboxylates (EDTA, DTPA)
viii. Pyridines (2,2′-bipyridine, 1,10-phenatholine, TPEN)
ix. Hydroxycarboxylates
x. Aroylhydrazones (PIH)
xi. Hydroxyquinolines (8-hydroxyquinoline)
xii. Hydroxypyrones (maltol, ethyl maltol)
xiii. Hydroxythiopyrones
   and molecules representing combinations of these chemical classes.

N-Hydroxy-6-octyloxypyridine-2(1H)one, ethanolamine salt, (HP-101) as supplied from Arch Chemicals, Inc., is part of the N-Hydroxypyridones. The N-Hydroxypyridones have alkyl ether substitutions at the 6-position as free acids, ethanolamine salts and metal salts such as zinc, N-Hydroxy-6-octyloxypyridine-2(1H)one, zinc salt. The alkyl ether substituent is from 2-22 carbons in length, either linear or branched.

For the zinc salts of materials such as EDDHA and EDDHMA, from Akzo-Nobel, the stoichiometry may be 1:1 zinc to ligand or 2:1. The chelating agents EDDHA, EDDHMA are intended to cover all their isomeric forms. Non-limiting examples of chelating agents covered by the term EDDHA include o,o-EDDHA (ethylenediamine-N,N′-di(2-hydroxyphenyl acetic acid), and o,p-EDDHA—ethylenediamine-N-(2-hydroxyphenyl acetic acid)-N′-(4-hydroxyphenyl acetic acid) and examples of the chelating agent EDDHMA include o,o-EDDHMA—ethylenediamine-N,N′-di(2-hydroxy-4-methylphenyl acetic acid), and o,o′-EDDHMA—ethylenediamine-N-(2-hydroxy-4-methylphenyl acetic acid)-N′-(2-hydroxy-6-methylphenyl acetic acid).

### B. Pyrithione or a Polyvalent Metal Salt of Pyrithione

In an embodiment, the present may comprise pyrithione or a polyvalent metal salt of pyrithione. Any form of polyvalent metal pyrithione salts may be used, including platelet and needle structures. In an embodiment, salts for use herein include those formed from the polyvalent metals magnesium, barium, bismuth, strontium, copper, zinc, cadmium, zirconium and mixtures thereof, and in a further embodiment, zinc. In a further embodiment, for use herein is the zinc salt of 1-hydroxy-2-pyridinethione (known as “zinc pyrithione” or “ZPT”); in yet a further embodiment, ZPT in platelet particle form, wherein the particles have an average size of up to about 20 μm, and in an embodiment have an average size of up to about 5 μm, and yet in a further embodiment have an average size of up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

It is further contemplated that when ZPT is used as the anti-microbial particulate in the anti-microbial compositions herein, that an additional benefit of hair growth or re-growth may be stimulated or regulated, or both, or that hair loss may be reduced or inhibited, or that hair may appear thicker or fuller.

Zinc pyrithione may be made by reacting 1-hydroxy-2-pyridinethione (i.e., pyrithione acid) or a soluble salt thereof with a zinc salt (e.g. zinc sulfate) to form a zinc pyrithione precipitate, as illustrated in U.S. Pat. No. 2,809,971.

Embodiments include from about 0.01% to about 5% of a pyrithione or polyvalent metal salt of a pyrithione; and from about 0.01% to about 5% of an iron chelator; more in an embodiment, each from about 0.1% to about 2%.

In embodiments having a pyrithione or polyvalent metal salt of pyrithione, the ratio of iron chelator to pyrithione or a polyvalent metal salt of pyrithione may be in the range of 1:10 to 10:1.

### C. Hydrophobicity

In one embodiment of the present invention, the iron chelator is hydrophobic. Sufficient hydrophobicity is required to increase the probability of the material traversing the cell membrane of the fungus to exert its effect. AlogP can be a measure of material hydrophobicity, which is calculated based on a chemical fragment approach. The more positive the number, the more hydrophobic the molecule, a minimum of which is believed to be needed to enhance membrane permeability. Intrinsic hydrophobicity can be calculated as AlogP for the isolated molecule. Materials with charged groups tend to be much less hydrophobic, often even becoming highly water soluble.

In the case where molecules are charged (especially anionic) and tend not to be hydrophobic enough to be cell permeable, an approach is to neutralize the charge by making certain metal ion salts, resulting in increased hydrophobicity and membrane permeability. The algorithms for calculating log P are not effective for metal salts and thus such differences must be measured. For sufficient hydrophobicity of a parent anionic material, the metal-ligand ratio should be sufficient to result in a charge neutral complex salt. Further, the metal used should be such that it can easily be replaced by metals such as iron once entering the cell. Desirable metal salts, then, should have a lower affinity for the ligand than iron; zinc salts are particularly preferred in this regard, though other salts are possible as long as they bind less strongly than iron.

In an embodiment of the present invention, a personal care composition comprises an iron chelator wherein the iron chelator is a metal salt. In a further embodiment of the present invention, an iron chelator is a metal salt selected from the group consisting of zinc salts, calcium salts, silver salts, nickel salts, magnesium salts, barium salts, bismuth salts and mixtures thereof. And in a further embodiment of the present invention, an iron chelator is a metal salt of zinc.

In a further embodiment of the present invention, the iron chelator has an AlogP value of greater than or equal to 0.4, and in an embodiment has an AlogP value of greater than or equal to 0.5, and in a further embodiment has an AlogP value of greater than or equal to 0.6, and in yet a further embodiment has an AlogP value of greater than or equal to 0.7,

### D. Iron Affinity—log K₁

The strength of the association between a ligand and metal, in this case iron, can be termed iron affinity. Without being bound by theory, materials that meet a minimum iron affinity have the potential to disrupt iron cellular metabolism thereby increasing anti-microbial activity when combined with other materials that are stressing cellular physiology.

Affinity between a metal (M) and ligand (L) can be measured by the stepwise association constant, K₁ which describes the following equilibrium:

$$M + L ⇌ ML , {K}_{1} = \frac{[ ML ]}{[ M ] [ L ]}$$

The affinity constant is conveniently expressed as the logarithm (log K₁) and the larger the magnitude of this number, the stronger the association between the metal (iron ions in this case) and ligand.

In an embodiment of the present invention, a log K₁>3.5 for demonstrating anti-fungal activity, in a further embodiment a log K₁>3.7, and in yet a further embodiment a log K₁>3.8.

### E. Intrinsic Anti-Malassezia Potency—IC50

The IC50 value is the inhibitor concentration that causes growth inhibition so that the culture optical density has increased ½ as much as that of the untreated control.

To measure the growth inhibition properties of test materials against Malassezia, we carried out the following protocol. We cultured Malassezia furfur in 100 ml mDixon medium (per one liter: 36 g malt extract (Difco 0186-17), 20 g ox bile (Fluka 70168), 10 ml Tween 40 (Aldrich 27435-6), 6 g peptone (Difco 0118-17), 2 ml oleic acid (Baker 2114-01), and 2 ml glycerol (Sigma G-7893). The pH is adjusted to 6.0 using 1N HCl (Baker 562-2). The media is autoclaved, and then 5 ml 0.1 g/ml chloramphenicol is added.

Starting cultures are prepared by incubating with shaking at 31°. Cells (5 ml of culture) are collected by centrifugation and suspended in 50 ml fresh mDixon medium. Cells (290 μl) are mixed with inhibitor (10 μl) in a deep well polypropylene plate (Beckman 267007) and sealed with a semipermeable membrane (Excel Scientific BS-25). The plates are shaken vigorously on a Heidolph Titramax 100 overnight at 31°. To increase the humidity, water-soaked cotton batting is placed over the plates. Some (200 μl) of the culture is transferred to a Costar 3596 plate, and the optical density (600 nm) is measured.

As a control, the optical density of untreated M. furfur is measured after overnight incubation. As another control, the optical density of the starting culture is measured. The difference between these optical densities represents the amount of growth of M. furfur.

TABLE 1

| | | | | Intrinsic |
|---|---|---|---|---|
| | | | | Anti- |
| | | Iron Affinity, | | Malassezia |
| | Hydrophobicity, | log K₁** | | Potency |
| Material | ALogP* | Fe²⁺, pH = 7.4 | | (IC50, μM) |
| Zinc Pyrithione | 1^{§} | 4.3 | (c) | 15.0 |
| 5,7-dichloro-8-hydroxyquinoline | 3.1 | 7.2 | (b) | 23.4 |
| N-Hydroxy-6-octyloxypyridine- | 3.5 | 5.4 | (d) | 54.6 |
| 2(1H)one, ethanolamine salt | | | | |
| (HP-101, Arch Chemical) | | | | |
| 1,5-Diphenyl-1,2,4,5-tetraazapent- | 5.0 | 6.5 | (a) | 62.5 |
| 1-en-3-thione | | | | |
| (Diphenylthiocarbazone, | | | | |
| Dithizone) | | | | |
| 1-Hydroxy-4-(1- | 1.9 | 4.9 | (c) | 65 |
| methylethyl)cyclohepta-3,5,7-trien- | | | | |
| 2-one (beta-Isopropyltropolone) | | | | |
| 5-Chloro-8-hydroxy-7- | 3.0 | 7.0 | (b) | 65.5 |
| iodoquinoline (Clioquinol) | | | | |
| N,N,N′,N-tetrakis(2-pyridylmethyl) | 3.1 | 14.2 | (a) | 71 |
| ethylenediamine (TPEN) | | | | |
| 2,2′: 6′,2″-terpyridine | 3.1 | 5.0 | (c) | 86 |
| Pyrithione | 0.7 | 4.3 | (c) | 90.0 |
| 8-hydroxyquinoline | 1.8 | 4.9 | (c) | 137.8 |
| 1,10-Phenanthroline | 2.2 | 5.9 | (a) | 166.5 |
| 1-(2-Thiazoylazo)-2-naphthol | 3.9 | 6.6 | (c) | 250 |
| trans-1,2- | −6.3 | 14.0 | (a) | 500 |
| Cyclohexylenedinitrilotetraacetic | | | | |
| acid (CDTA) | | | | |
| kojic acid | −1.1 | 3.7 | (c) | 704.0 |
| 3-Hydroxy-1,2-dimethyl-4(1H)- | −0.1 | 3.9 | (c) | 719.0 |
| pyridone (Deferiprone) | | | | |
| maltol | −0.2 | 6.4 | (c) | 793.0 |
| 2,9-dimethyl-1,10-phenanthroline | 2.8 | 3.3 | (c) | >480 |
| 1,4,7,10,13,16- | −2.4 | 13.3 | (e) | >500 |
| Hexaazacyclooctadecane | | | | |
| ([18]aneN6) | | | | |
| 1,4,8,11- | −13.5 | 9.9 | (a) | >500 |
| Tetraazacyclotetradecane- | | | | |
| N,N′,N″,N″′-tetraacetic acid (TETA) | | | | |
| 1-Hydroxy-1,2-dihydropyridin-2- | −0.3 | 4.2 | (c) | >500 |
| one | | | | |
| 2-(2-aminoethyl)-pyridine | 0.2 | 5.2 | (d) | >500 |
| beta-Alanyl-L-histidine | −4.5 | 1.3 | (c) | >500 |
| (L-Carnosine) | | | | |
| Ethylenediiminodi-2-butanoic acid | −5.0 | 6.3 | (c) | >500 |
| L-2-Amino-3-phosphopropanoic | −4.1 | 2.3 | (c) | >500 |
| acid (Phosphoserine) | | | | |
| N,N-Bis(2-hydroxyethyl)glycine | −4.3 | 3.6 | (c) | >500 |
| (Bicine) | | | | |
| Salicylaldoxime | 1.4 | 0.7 | (d) | >500 |
| Salicylic acid | 1.2 | −3.0 | (e) | >500 |
| Ethylenediaminetetraacetic acid | −7.7 | 12.2 | (a) | >500 |
| (EDTA) | | | | |
| 4-(2-thiazolylazo)-resorcinol | 2.7 | 3.2 | (a) | >900 |

^{§}Measured, not calculated
*Calculated by published methods, see A K Ghose J Phys Chem A 1998, 102, 3762.
**Data obtainerd from sources as marked:
(a) NIST Standard Reference Database 46, Version 8.0: NIST Critical Selected Stability Constants of Metal Complexes Database. Arthur E. Martell & Robert M. Smith, 2004.
(b) IUPAC Stability Constant Database.
(c) Data interpolated from the correlation between Zn²⁺ and Fe²⁺ data from sources (a) and (b) with a a regression equation of y = 0.8646x − 0.2482 with R² = 0.91.
(d) Data interpolated from the correlation between Fe³⁺ and Fe²⁺ data from sources (a) and (b) with a regression equation of y = 0.6637x − 2.056 with R² = 0.90.
(e) Data interpolated from the correlation between Cu²⁺ and Fe²⁺ data from sources (a) and (b) with a regression equation of y = 0.7003x − 1.0113 with R² = 0.78.

### F. Fractional Inhibitor Concentration (FIC)

Fractional Inhibitor Concentration (FIC) is a conventional methodology for evaluating antimicrobial interactions. In an embodiment of the present invention, an FIC is used to determine combinatorial effects of two chemicals on anti-Malassezia activity in a tissue culture setting.

### I. Method Overview:

1. Each FIC assay is run in duplicate.
2. Low levels of M. furfur 7982 cells are inoculated into each well of a 96 well plate.
3. Chemical #1 (in our assays, this is always ZPT) is titrated (2× concentration reductions) from right to left across each row of the plate, starting at 12.5 ppm final concentration and ending at 0 ppm.
4. Chemical #2 is titrated (2× concentration reduction) down each column of the plate, starting at either 400 ppm or 800 ppm final concentration (depending on potency) and ending at 0 ppm.
5. Plates are incubated at 33° C., 60% RH for 2 days with agitation.
6. Optical densities (OD's) are then determined using a spectrophotometer.

### II. Data Analysis:

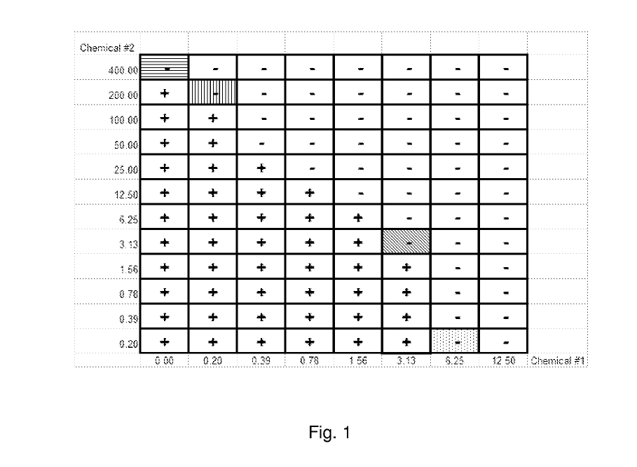

1. Inhibition of growth is determined if the OD<=0.6 (this indicates that growth in the well did not achieve at least 50% of the maximal level possible), and the well is labeled with a ‘−’.
2. Lack of inhibition of growth is determined if the OD>0.6, and the well is labeled with a ‘+’.
3. The lowest concentration value of chemical #1, both by itself and with the lowest concentration of chemical #1 which boosts chemical #2's potency, that inhibit growth are determined (see FIG. 1).
4. Likewise, the lowest concentration of chemical #2, both by itself and with the lowest concentration of chemical #2 that boosts chemical #1's potency, that inhibit growth are determined (see FIG. 1).
5. The FIC value for the duplicate plates is determined using the calculations below:

$$FIC = \frac{lowest inhibitory dose of Chemical \#1 by itsef}{\begin{matrix}inhibitory dose of Chemical \#1 in a well also \\ contains Chemical \#2 also contains Chemical \#1\end{matrix}} + \frac{lowest inhibitory dose of Chemical \# 2 by itselflowest}{lowest inhibitory dose of Chemical \#2 in a well that}$$

$$ave FIC VALUE = {( FIC value for plate 1 + FIC value for plate 2 )}_{2}$$

6. The average FIC value is then determined, and the combinatorial effect of the two chemicals is classified according to the chart below:

| ave FIC VALUE | Combinatorial Effect |
|---|---|
| <=0.5 | Synergistic |
| >0.5-1.0 | Additive |
| >1.0-<=4.0 | Indifferent |
| >4 | Antagonistic |

Per the instructions noted in section II.1, in FIG. 1, each well has been labeled either ‘+’ or ‘−’. The horizontal lined well indicates the lowest value of chemical #2 by itself (read to the left as 400.00). The diagonally lined well indicates the lowest value of chemical #2 that provides a boost to chemical #1's potency (read to the left is 3.13). The dotted well indicates the lowest value of chemical #1 by itself (read downward as 6.25). The vertical lined well indicates the lowest value of chemical #1 that provides a boost to chemical #2's potency (read downward as 0.20).
Per the instructions noted in section II.5, the FIC value is calculated by the following equation:

(0.20/6.25)+(3.13/400.00)=0.04

Per the instructions noted in section II.1, this plate can be been run in duplicate, and here it is assumed to have given the same result.

Per the instructions noted in section II.5, the average FIC value is calculated by the following equation:

(0.04+0.04)/2=0.04

Per the instructions noted in section II.6, given the average FIC value of 0.04, the combinatorial effect of these two chemicals is determined to be synergistic.

TABLE 2
Examples of Additivity and Synergy for Chelators with ZPT

| Material | FIC* |
|---|---|
| EDDHMA, Zinc Salt (1) | 0.1 |
| Maltol, Zinc salt | 0.1 |
| EDDHA, Zinc Salt (1) | 0.2 |
| Benzohydroxamic acid, Zinc Salt | 0.3 |
| Thiomaltol, Zinc salt | 0.5 |
| N-Hydroxy-6-octyloxypyridine-2(1H)one, ethanolamine | 0.62 |
| salt (HP-101) (2) | |
| N-Hydroxy-6-octyloxypyridine-2(1H)one, zinc salt | 0.7 |
| (HP-100 Zn) (2) | |
| 1,2-Dimethyl-3-hydroxy-4-pyridone, Zinc salt | 0.5 |
| Thiomaltol | 0.8 |
| N,N-Naphthaloylhydroxylamine | 0.8 |

(1) From Akzo-Nobel
(2) From Arch Chemicals, Inc.
*Fractional Inhibitory Concentration wherein FIC ≦ 0.5 represents synergistic activities and 0.5 < FIC ≦ 1.0 represents additivity (FIC's above 1.0 are either indifferent or antagonistic). Reference: R. Bharadwaj et al Indian J Pharmacol 2003, 35, 220.

The hydrophobicity and iron affinity data of Table 1 are arranged in approximate order of decreasing intrinsic anti-fungal potency (measured as IC₅₀). The intrinsic anti-fungal potency is being used as a predictor of the potential to have either synergistic or additive anti-microbial benefits when combined with another anti-fungal agent.

The data of Table 1 clearly show both hydrophobity and iron affinity parameters must meet certain minimal criteria to result in appreciable independent anti-fungal activity. For example, the chemically related 1,10-phenanthroline and 2,9-dimethyl phenanthroline have substantially different intrinsic anti-fungal potencies that can be interpreted as being due to 2,9-diphenyl phenanthroline having a much lower iron affinity than the parent phenanthroline molecule.

Note that very common iron chelators such as EDTA are observed not to be effective, this is likely due to the very low hydrophobicity which, without being bound by theory, likely limits the permeability into the cell, where the site of action of iron binding is thought to occur.

When evaluating the additivity and synergism data of Table 2, it is clear that there can be very strong interactions with, in this example, zinc pyrithione to increase anti-fungal potency of the combination. In these examples, it becomes clear that some materials can be substantially improved by making certain metal salts of the materials. For example, maltol (from Table 1) is too hydrophilic to be an effective anti-fungal material independently. However, its zinc salt is considerably more hydrophobic (cannot be calculated by the A log P method) enabling it to potentiate the activity of zinc pyrithione (see table 2, FIC of 0.1).

Anionic iron binding materials have the potential, in general, to be made more hydrophobic, and thereby increase cellular permeability, by making certain metal salts of these materials. While zinc is most preferred, other metals, such as calcium, magnesium and barium are possible.

In an embodiment of the present invention, the combination of an iron chelator and pyrithione or a polyvalent metal salt of a pyrithione has a fractional inhibitor concentration of less than or equal to 1. And an iron chelator in combination with pyrithione or a polyvalent metal salt of a pyrithione has a fractional inhibitor concentration of less than or equal to 1. In a further embodiment of the present invention, the combination of an iron chelator and pyrithione or a polyvalent metal salt of a pyrithione has a fractional inhibitor concentration of less than or equal to 0.5.

Additional information regarding fractional inhibitor concentration is found in AN INVITRO STUDY TO EVALUATE THE SYNERGISITC ACTIVITY OF NORFLOXACIN AND METRONIDAZOLE, R. Bharadwaj, et al., Indian J Pharmacol 2003, 35: 220-226.

In an embodiment of the present invention, the personal care composition may further comprise a surfactant. In a further embodiment, the composition of the present invention may include a detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance. Suitable anionic detersive surfactant components for use in the composition herein include those which are known for use in hair care or other personal care cleansing compositions. Nonlimiting examples of anionic surfactants are described in U.S. Pat. Nos. 2,486,921; 2,486,922; 2,396,278 and 3,332,880. The concentration of, for example, an anionic surfactant component in the composition should be sufficient to provide the desired cleaning and lather performance, and generally range from about 5% to about 50%. Non limiting examples of other anionic, zwitterionic, amphoteric or optional additional surfactants suitable for use in the compositions are described in McCutcheon's, Emulsifiers and Detergents, 2002 Annual, published by M. C. Publishing Co., and U.S. Pat. Nos. 3,929,678, 2,658,072; 2,438,091; 2,528,378.

In a further embodiment, the composition of the present invention may be in the form of a topical compositions, which includes a topical carrier. In an embodiment, the topical carrier is selected from a broad range of traditional personal care carriers depending on the type of composition to be formed. By suitable selections of compatible carriers, it is contemplated that such a composition is prepared in the form of daily skin or hair products including conditioning treatments, cleansing products, such as hair and/or scalp shampoos, body washes, hand cleansers, water-less hand sanitizer/cleansers, facial cleansers, deodorants and the like.

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10%.

Non-limiting examples of optional components for use in the composition include cationic polymers, cationic surfactants, conditioning agents (hydrocarbon oils, fatty esters, silicones), anti dandruff agents, suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, vitamins, minerals, herbal/fruit/food extracts, sphingolipids derivatives or synthetical derivative, and clay. Nonlimiting examples of the optional components are found in International Cosmetic Ingredient Dictionary, Fifth Edition, 1993, and CTFA Cosmetic Ingredient Dictionary, 3rd edition, edited by Estrin, Crosley, and Haynes, (The Cosmetic, Toiletry, and Fragrance Association, Inc., Washington, D.C. (1982)).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) an effective amount of a pyrithione or a polyvalent metal salt of a pyrithione;
b) an effective amount of an iron chelator or a material which chelates iron;
wherein the combination of the iron chelator and the pyrithione or a polyvalent metal salt of a pyrithione has a fractional inhibitor concentration of less than or equal to 1.

2. A personal care composition according to claim 1 wherein the iron chelator is hydrophobic.

3. A personal care composition according to claim 1 wherein the iron chelator has an AlogP value of greater than or equal to 0.4.

4. A personal care composition according to claim 1 wherein the iron chelator has a log K₁>3.5.

5. A personal care composition according to claim 1 wherein the pyrithione or polyvalent metal salt of pyrithione is zinc pyrithione.

6. A personal care composition according to claim 1 wherein the pyrithione or polyvalent metal salt of pyrithione is copper pyrithione.

7. A personal care composition according to claim 1 wherein the iron chelator is selected from the group consisting of materials having an affinity for iron ions in either the ferrous (iron II) or ferric (III) forms, materials having an affinity for iron ions in either the ferrous (iron II) or ferric (III) forming a denticity of two or higher, catechols and phenols, hydroxamates, thiohydroxamates, hydroxypyridones, hydroxythiopyridones, hydroxypyridinethiones, aminocarboxylates, pyridines, hydroxycarboxylates, aroylhydrazones, hydroxyquinolines, hydroxypyrones, hydroxythiopyrones and mixtures thereof.

8. A personal care composition according to claim 7 wherein the iron chelator is selected from group comprising natural chelators, synthetic chelators and mixtures thereof.

9. A personal care composition according to claim 1 wherein the iron chelator is a metal salt.

10. A personal care composition according to claim 9 wherein the iron chelator is a metal salt selected from the group consisting of zinc salts, calcium salts, silver salts, nickel salts, magnesium salts, barium salts, bismuth salts and mixtures thereof.

11. A personal care composition according to claim 10 wherein the iron chelator is a metal salt of zinc.

12. A personal care composition according to claim 1 the wherein the pyrithione or a polyvalent metal salt of a pyrithione is present from about 0.01% to about 5%.

13. A personal care composition according to claim 1 wherein the iron chelator is present from about 0.01% to about 5%.

14. A personal care composition according to claim 1 wherein the combination of the iron chelator and the pyrithione or a polyvalent metal salt of a pyrithione has a fractional inhibitor concentration of less than or equal to 0.5.
